(19) United States
(12) Patent Application Publication
(10) Pub. No.: US 2006/0083768 A1
(43) Pub. Date: Apr. 20, 2006

(54) Method of thickening a coating using a drug

(75) Inventors: Roger Labrecque, Londonderry, NH (US); Geoffrey Moodie, Hudson, NH (US); Suzanne Conroy, Dracut, MA (US); Lisa Rogers, Londonderry, NH (US); Joseph Ferraro, Londonderry, NH (US); Theodore Karwoski, Hollis, NH (US); Steve A. Herweck, Nashua, NH (US); Paul Martakos, Pelham, NH (US)

Correspondence Address: LAHIVE & COCKFIELD, LLP., 28 STATE STREET, BOSTON, MA, 02109, US

(73) Assignee: ATRIUM MEDICAL CORPORATION, Hudson, NH (US)

(21) Appl. No.: 11/238,564

(22) Filed: Sep. 28, 2005

Related U.S. Application Data
(60) Provisional application No. 60/613,745, filed on Sep. 28, 2004.
(60) Provisional application No. 60/613,808, filed on Sep. 28, 2004.

(51) Int. Cl. A61F 2/00; A61K 48/00

(52) U.S. Cl. 424/423; 514/44; 514/291; 514/449; 514/460; 514/548; 514/458

(57) ABSTRACT

A method for the provision of a coating on an implantable medical device results in a medical device having a bio-absorbable coating. The coating includes a bio-absorbable carrier component. In addition to the bio-absorbable carrier component, a dissolved therapeutic agent component can also be provided. The coated medical device is implantable in a patient to effect controlled delivery of the coating, including the dissolved therapeutic agent, to the patient.

### RELATED APPLICATIONS

This application claims priority to, and the benefit or, co-pending U.S. Provisional Application No. 60/613,745, Sep. 28, 2004, and co-pending U.S. Provisional Application No. 60/613808, filed Sep. 28, 2004, for all subject matter common to all applications. The disclosure of said provisional applications is hereby incorporated herein by reference in its entirety. This application also relates to co-pending U.S. patent application Ser. No. 11/______, (Attorney Docket No. ATA-426) and U.S. patent application Ser. No. 11/______, (Attorney Docket No. ATA-427), filed concurrently with this application on Sep. 28, 2005.

### FIELD OF THE INVENTION

The present invention relates to coatings and preparations of coatings for medical devices for the delivery of one or more biologically active agents, and more particularly, the present invention relates to increasing the viscosity of coatings capable of containing one or more biologically active components using a therapeutic agent.

### BACKGROUND OF THE INVENTION

Percutaneous transluminal coronary angioplasty (PTCA), also known as balloon angioplasty, is a technique widely used for treating intravascular diseases, such as atherosclerosis, and other vascular occlusions. PTCA involves the use of a balloon-tipped catheter inserted directly into the arteries and vessels of a subject until the occluded site is reached, whereupon the balloon is expanded. The inflation of the balloon forces the lumen open, allowing blood flow to be restored. However, while PTCA is effective in the short-term, approximately 30-50% of all cases of balloon angioplasty alone require follow-up angioplasty due to restenosis, or re-narrowing of the blood vessel or artery.

Restenosis is caused by three pathogenic factors: elastic recoil of the artery, late-stage remodeling of the artery and hyperproliferation of the smooth muscle cells of the artery. This hyperproliferation, called neointimal hyperplasia, occurs as a result of the body's natural response to the arterial injury caused by the PTCA procedure. Upon the deployment of the balloon catheter, small tears develop in the artery wall triggering an inflammatory response. Growth factors and cytokines produced during the inflammatory response activate smooth muscle cell proliferation and migration, which can form an obstructing neointima, which, in turn, leads to decreased blood flow through the artery.

Prevention of occlusive thrombus after PTCA can be accomplished by the administration of oral high-dose, systematic anti-platelet drug therapy in combination with aspirin. This course of action has been shown to limit early complications after PTCA by approximately 35%; however, serious bleeding complications and other side effects can occur. Additionally, an orally administered drug may not achieve the desired effect in the area of the body in which it is needed. Furthermore, success by oral medication depends entirely on patient compliance.

Currently, the only long term approach to preventing restenosis is by utilizing a medical device, such as a stent, as an arterial structural support. While deployment of a stent after PTCA effectively eliminates elastic recoil and counteracts arterial remodeling, in-stent restenosis is still a serious problem due to neointimal hyperplasia. Introduction and presence of the stent itself can create regions of trauma in the artery, causing the same inflammatory response as the PTCA procedure.

Stent-based drug delivery has been developed in an attempt to prevent in-stent restenosis. Local delivery of one or more therapeutic agents by the use of a drug-eluting stent shows promise as a solution to the problems of both early and late complications due to the PTCA procedure. A number of therapeutic agents have been studied for use with stents including anticoagulants (heparin, hirudin), anti-platelet agents (abciximab), anti-inflammatory drugs (dexamethasone), anti-migratory agents (batimastat) and anti-proliferative agents (sirolimus, paclitaxel, actinomycin D).

Typically, the drug-eluting stent is coated with a polymeric material. The polymer may improve the quality of the stent by strengthening it or by smoothing the surface of the stent to minimize damage to the endothelium. In addition, the polymer may serve as the component used to adhere the therapeutic agent to the stent itself. Furthermore, the polymer may serve as the vehicle for local drug delivery, for example, by serving as a drug depot and/or degrading such that the drug is released to the desired area. There are substantial concerns, however, regarding the lack of bio-compatibility of polymer stent coatings. An assortment of both biodegradable and non-biodegradable polymers have been shown to induce an inflammatory response within the coronary artery, including neointimal thickening (see, for example, van der Giessen, et al. Circulation 1996;94: 1690-1697; De Schreerder, et al Atherosclerosis 1995; 114:105-114, incorporated herein by reference in their entirety).

There is a need, then, to produce a drug-eluting stent without a polymeric coating. However, a coating is needed to replace the functions performed by the polymer. For example, a coating is needed to dissolve the therapeutic agent, as well as serve as the element to adhere the therapeutic agent to the stent. In addition, the coating would also be the vehicle for local delivery for the therapeutic agent.

U.S. Patent Application Publication No. 20030191179 is directed to a method of administration of paclitaxel formulated with a vitamin E derivative. The composition for delivery of paclitaxel comprises paclitaxel, a solvent, and a pharmaceutically acceptable, water-miscible solubilizer which has the general structure of R₁COOR₂, R₁CONR₂ and R₁COR₂, wherein R₁ is a hydrophobic C₃-C₅₀ alkane, alkene or alkyne, and R₂ is a hydrophilic moiety. The publication indicates that the solubilizer can be an esterified fatty acid or alpha-tocopherol polyethylene glycol succinate, which is a water-miscible derivative of alpha-tocopherol.

PCT Application Publication No. WO 99/25336 is directed to a method for preventing restenosis in a patient by administering a prophylactically effective amount a composition of a tocotrienol or a mixture of tocotrienols. The publication is additionally directed to a method for preventing restenosis in a patient undergoing arterial angioplasty by coating the external surface of the angioplastic balloon with a composition containing tocotrienols. These compositions are prepared by combining one or more tocotrienols with an acceptable carrier. Suitable carriers include glycols, parabens, glycerin, alcohols, petrolatum oils and waxes. The '336 patent application treats the tocotrienols as the therapeutic agent for treating restenosis that is contained within a carrier component.

U.S. Patent Application Publication No. 20040156879 is directed to a method of manufacturing oxidation resistant medical implants and, in particular, antioxidant-doped medical devices containing cross-linked polymers. The method includes doping consolidated polyethylene, such as ultra-high molecular weight polyethylene (UHMWPE), with anti-oxidants before, during or after crosslinking the consolidated polyethylene. The patent application indicates that the doping of the consolidated polyethylene can be carried out by diffusion of an antioxidant. Suitable antioxidants include alpha- and delta-tocopherols; propyl, octyl, or dedocyl galates; lactic, citric, and tartaric acids and their salts; orthophosphates, tocopherol acetate and vitamin E. The doping method involves soaking the consolidated UHMWPE in the antioxidant or in a solution of the antioxidant when the antioxidant is dissolved in ethanol. The '879 patent application calls for the use of a consolidated polyethylene in the preparation of the described medical devices.

U.S. Pat. No. 6,833,004 is directed to a stent with a biologically and physiologically active substance stably loaded onto the stent main body such that the biologically and physiologically active substance does not decompose or degrade, but, once implanted, the biologically and physiologically active substance undergoes sustained release. The stent includes a main body with a sustained release coating made up of two layers: a layer containing the biologically and physiologically active substance and a polymer layer formed on top of the biologically and physiologically active substance layer. If the biologically and physiologically active substance is unable to adhere to the wire member constituting the stent main body, then the layer containing the biologically and physiologically active substance can be supplemented with an additional component which will impart tackiness to the biologically and physiologically active substance. For example, if the biologically and physiologically active substance is a fat soluble substance, the additional component is a low molecular weight higher fatty acid having a molecular weight of up to 1000, such as a fish oil, a vegetable oil or a fat soluble vitamin such as vitamin A or vitamin E. The medical device in the '004 patent is treated with a polymeric layer after the application of the biologically and physiologically active substance, with or without the additional component.

U.S. Pat. No. 6,117,911 is directed to the use of compounds and different therapies for the prevention of vascular and non-vascular pathologies. The '911 patent discusses the possibility of using many different types of delivery methods for a therapeutic agent or agents to prevent various vascular and non-vascular pathologies. One such approach is described as providing a method of preventing or treating a mammal having, or at risk of developing, atherosclerosis, including administering an amount of a combination of aspirin or an aspirinate and at least one omega-3 fatty acid, wherein said amount of omega-3 fatty acid is effective to maintain or increase the level of TGF-beta so as to provide a synergistic effect with a therapeutic compound to inhibit or reduce vessel lumen diameter diminution. As such, the patent discusses some of the therapeutic benefits of primarily systemic administration of omega-3 fatty acids, such as those found in fish oil, to affect TGF-beta levels when a therapeutic agent is combined with aspirin or aspirinate. That is, the dose or concentration of omega-3-fatty acid required to increase the level of TGF-beta is significantly greater, requiring long term systemic delivery.

U.S. Patent Application No. 20030077310 is directed to coated stents, methods of making coated stents and methods of using coated stents, wherein the coating contains unreacted HMG-CoA reductase inhibitor in combination with a carrier. The carrier can either be polymeric or non-polymeric. When the carrier is non-polymeric, it can be a C6 to C18 fatty acid, a bio-compatible wax, oil or gel, or a mixture of one or more of a wax, an oil, a gel, and a fatty acid. The non-polymeric liquid carrier can also be a hydrophobic liquid, such as a C4-C36 fatty acid, for example, oleic or stearic acid, or an oil, such as peanut oil, cottonseed oil, mineral oil, or other low molecular weight oils (C4-C36).

U.S. Pat. No. 6,610,035 is directed to an implantable medical device with a bi-layer lubricious coating. The first layer consists of a hydrophilic polymeric hydrogel layer which can swell or dissolve upon exposure to an aqueous environment. The second layer of the coating comprises a hydrophobic coating, which can be silicone based or a naturally occurring composition including olive oil, paraffin oil, corn oil, sesame oil, fish oil, and vegetable oil. The medical devices described by the '035 patent are treated with a hydrophilic polymer gel prior to the addition of a hydrophilic coating.

U.S. Patent Application No. 20030083740 is directed to a method of forming liquid coatings for medical devices made from biodegradable materials in liquid, low melting solid or wax forms which further degrade upon implantation without producing harmful fragments. The liquid coatings additionally can contain biologically active compounds which are released upon degradation of the coatings after implantation. The carrier component of the coating composition can be hydrophobic, bio-compatible and either polymeric or non-polymeric. Suitable non-polymeric carrier components comprise vitamin E or its derivatives, oleic acid, stearic acid, mineral oil, peanut oil, or cottonseed oil, alone or in combination.

U.S. Pat. No. 6,610,068 is directed to a catheter device with a guide member lumen filled with a lubricious material. The method of filling the guide member lumen with a lubricious material eliminates the need for flushing the catheter device before and during surgical procedures and provides a lubricant for easy maneuvering of the catheter over the guide member. The '068 patent indicates that the lubricious material can include both hydrophobic and hydrophilic materials. Specifically, the hydrophobic materials can include silicone based lubricants, glycerine, olive oil, cottonseed oil, peanut oil, fish oil, vegetable oil, sesame oil, and vitamin E. Vitamin E, if used, can also act as an antioxidant. The antioxidant capability of vitamin E improves the long term stability of the lubricious coating.

PCT Application Publication No. WO 02/100455 is directed to ozonated medical devices and methods of using ozone to prevent complications from indwelling medical devices. The application discusses having the ozone in gel or liquid form to coat the medical device. The ozone can be dissolved in olive oil, or other types of oil, to form a gel containing ozone bubbles, and the gel applied to the medical device as a coating. The application later asserts a preference for the gel or other coating formulation to be composed so that the ozone is released over time. However, there is no indication in the application as to how a slow controlled release of ozone can be affected. There is no enablement to a long term controlled release of ozone from the olive oil gel, however, there is mention of use of biocompatible polymers to form the coating that holds and releases the ozone. Other drugs are also suggested for combination with the ozone for delivery to a targeted location. The application later describes different application methods for the coating, including casting, spraying, painting, dipping, sponging, atomizing, smearing, impregnating, and spreading.

A paper entitled “Evaluation of the Biocompatibility and Drug Delivery Capabilities of Biological Oil Based Stent Coatings”, by Shengqiao Li of the Katholieke Universiteit Leuven (incorporated herein by reference in its entirety), discusses the use of biological oils as a coating for delivering drugs after being applied to stents. Three different coatings were discussed, a glue coating (cod liver oil mixed with 100% ethanol at a 1:1 ratio), a vitamin E coating (97% vitamin E oil solution mixed with 100% ethanol at a 1:1 ratio), and a glue+vitamin E coating (cod liver oil and 97% vitamin E oil solution mixed with 100% ethanol at a 1:1 ratio). Bare stents and polymer coated stents, along with stents having each of the above coatings, were implanted into test subjects, and analyzed over a four week period. At the end of the period, it was observed that the bare stents and polymer coated stents resulted in some minor inflammation of the tissue. The main finding of the study was that the glue coatings have a good biocompatibility with coronary arteries, and that the glue coating does not affect the degree of inflammation, thrombosis, and neointimal proliferation after endovascular stenting compared with the conventional stenting approach. A further hypothesis asserted was that the oil coating provided lubrication to the stent, thus decreasing the injury to the vascular wall.

The study went on to analyze the drug loading capacity of biological oil based stent coatings. Balloon mounted bare stents were dip-coated in a biological oil solution with the maximal solubilizable amount of different drugs (a separate drug for each trial), and compared with polymer coated, drug loaded, stents. According to the release rate curves, there was a clear indication that drug release was fast in the first 24 hours with more than 20% of the drug released, for the oil based coatings. The release rate after the first 24 hours was much slower, and continued for a period up to about six weeks.

Another aspect of the study looked at the efficacy of drug loaded biological stents to decrease inflammation and neointimal hyperplasia in a porcine coronary stent model. In this part of the study, glue or modified glue (biological oil) coated stainless steel stents were loaded with different drugs. The result was that the characteristics of the particular drug loaded onto the stent were the major factor to the reduction of restenosis, and the biological oil did not have a major impact on either causing or reducing inflammation.

A further comment indicated that in the studies comparison was made between biological oil based drug loaded stents and bare stents to find differences in inflammation, injury, and hyperplasia. Inflammation, injury, and neointimal hyperplasia resulted in in-stent area stenosis. Any anti-inflammation observed was the result of the particular drug loaded on the stent, regardless of biological oil, or polymer, coating.

A paper entitled “Addition of Cytochalasin D to a Biocompatible Oil Stent Coating Inhibits Intimal Hyperplasia in a Porcine Coronary Model” by Koen J. Salu, et al (Coronary Artery Disease 2003;14:545-555, incorporated herein by reference in its entirety) discusses the use of a natural oil as a stent coating and the efficacy of using a therapeutic agent combined with the natural oil coating for the prevention of restenosis. The study first performed a histopathological evaluation of eicosapentaenoic acid oil coated stents compared with bare, uncoated stents. A series of stents coated in eicosapentaenoic acid oil and bare stents were implanted into test subjects and were analyzed after 5 days and again after 4 weeks. In all cases, there was an identical tissue response between the bare stents and the eicosapentaenoic acid oil coated stents. It was also found that the oil-coating did not elicit a hyperproliferative or inflammatory response. The study proposed that the lack of inflammation or hyperproliferation of the coated stent was due to the properties of eicosapentaenoic acid, which exerts anti-inflammatory effects and inhibit vascular smooth muscle cell proliferation in vitro.

Another aspect of the study compared eicosapentaenoic acid oil coated stents with stents coated with a therapeutic agent solubilized in eicosapentaenoic acid oil. The therapeutic agent examined was cytochalasin D, a lipophilic, cell-permeable fungal metabolite that inhibits the polymerization of actin into microfilaments. The results of this aspect of the study indicated that the inclusion of the therapeutic agent led to 39% less intimal hyperplasia and 38% less area stenosis when compared to the control group.

PCT Application Publication No. WO 03/039612 is directed to an intraluminal device with a coating containing a therapeutic agent. The publication describes coating an intraluminal device with a therapeutic agent comprised of a matrix that sticks to the intraluminal device. The matrix is formed of a bio-compatible oil or fat, and can further include alpha-tocopherol. The publication further indicates that an oil or fat adheres sufficiently strongly to the intraluminal device so that most of the coating remains on the intraluminal device when it is inserted in a body lumen. The publication further states that the oil or fat slows the release of the therapeutic agent, and also acts as an anti-inflammatory and a lubricant. The publication goes on to indicate that the oil or fat can be chemically modified, such as by the process of hydrogenation, to increase their melting point. Alternatively, synthetic oils could be manufactured as well. The oil or fat is further noted to contain fatty acids.

The '612 publication provides additional detail concerning the preferred oil or fat. It states that a lower melting point is preferable, and a melting point of 0° C. related to the oils utilized in experiments. The lower melting point provides a fat in the form of an oil rather than a wax or solid. It is further stated that oils at room temperature can be hydrogenated to provide a more stable coating and an increased melting point, or the oils can be mixed with a solvent such as ethanol. Preferences were discussed for the use of oils rather than waxes or solids, and the operations performed on the fat or oil as described can be detrimental to the therapeutic characteristics of some oils, especially polyunsaturated oils containing omega-3 fatty acids.

The above-described references do refer to the use of oils and fats as a drug delivery platform. There is indication that the coatings described in the above references are bio-absorbable, while also providing the release of biologically active components, such as drugs. Additionally, many of the above-described patents and patent applications require the use of a polymeric material, which serves as either a base upon which a drug coating is applied, a substance mixed in with the drug to form the coating, or a top coating applied over a previously applied drug coating to control the release of the drug. However, there is no realization of the difficulty of using an oil having its own therapeutic characteristics for the solubilization and release of a therapeutic agent.

U.S. Pat. No. 6,761,903 is directed to pharmaceutical compositions capable of solubilizing therapeutically effective amounts of therapeutic agents. The patent discusses pharmaceutical compositions having a carrier and a therapeutic agent, as well as pharmaceutical composition comprising an oil soluble vitamin and a carrier. The carrier for both pharmaceutical compositions includes a triglyceride in combination with at least two surfactants, wherein one of the surfactants is hydrophilic. Suitable triglycerides include a number of oils, including fish oil, while suitable surfactants include a variety of fatty acid ester derivatives and polymers, transesterified products of oils and alcohols, mono- and diglycerides, sterols, sterol derivatives, polymer glycol alkyl ethers and alkyl phenols, sugar esters, POE-POP block co-polymers, and ionic surfactants, such as the salts of fatty acids and bile salts. The '903 patent further discusses the use of oil-soluble vitamins for improving the solubility and stability of therapeutic agents in the pharmaceutical compositions, and that there may be improved absorption or permeability of the therapeutic agents across an absorption barrier, such as a mucosal membrane.

The above-referenced patent does describe the use of an oil based pharmaceutical composition capable of solubilizing therapeutic agents. However, the '903 patent always requires the use of a hydrophilic surfactant and does not indicate the use of the pharmaceutical compositions described for medical devices.

What is desired is a bio-absorbable delivery agent with increased viscosity having non-inflammatory and other therapeutically advantageous characteristics for the delivery of a therapeutic agent to body tissue.

### SUMMARY OF THE INVENTION

There is a need for a bio-absorbable coating of increased viscosity for application to an implantable medical device for therapeutic purposes. The present invention is directed toward further solutions to address the need for increasing the viscosity of coatings capable of containing one or more biologically active components using a therapeutic agent.

In accordance with one aspect of the present invention, a method of increasing the viscosity of an oil based composition is provided. Accordingly, the steps of the method include providing the oil-based composition comprising at least one fatty acid and combining the oil-based composition with one or more therapeutic agents in an amount sufficient to increase viscosity of the oil based composition.

In accordance with one aspect of the present invention, a coating for a medical device is provided. Accordingly, the coating for the medical device is formed at least in part of an oil comprising at least one fatty acid component and at least one therapeutic agent component. In one embodiment, the therapeutic agent component is combined with the composition in an amount sufficient to increase a viscosity of the composition to a viscosity measurement greater than a viscosity measurement of the oil prior to combination with at least one therapeutic agent.

In accordance with one aspect of the present invention, the one or more fatty acids of the oil-based composition can include arachidic acid, gadoleic acid, arachidonic acid, eicosapentaenoic acid (EPA), docosahexaenoic acid (DHA), butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, analogs and pharmaceutically acceptable salts thereof.

In accordance with one aspect of the present invention, the therapeutic agent can include an antioxidant, an anti-inflammatory, an anti-coagulant, a drug to alter lipid metabolism, an anti-proliferative, an analgesic, an anti-neoplastic, an anti-fibrotic, an immunosuppressive, a tissue growth stimulant, a functional protein/factor delivery agent, an anti-infective agent, an imaging agent, an anesthetic, a chemotherapeutic agent, a tissue absorption enhancer, an anti-adhesion agent, a germicide, an antiseptic, a proteoglycan, a GAG, a gene delivery agent (polynucleotide), an analgesic, a polysaccharide (e.g. heparin), anti-migratory agents, pro-healing agents, and ECM/protein production inhibitors, or a combination thereof. Furthermore, the therapeutic agent can be rapamycin, melatonin, paclitaxel, a protein kinase C inhibitor, cerivastatin, cilostazol, fluvastatin, lovastatin, pravastatin or derivatives, prodrugs, analogs and pharmaceutically acceptable salts thereof.

In accordance with one aspect of the present invention, the oil-based composition can further comprise a vitamin E compound. Accordingly, the vitamin E compound can include alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, vitamin E TPGS, mixed tocopherols, derivatives, analogs and pharmaceutically acceptable salts thereof. It should also be noted that other antioxidants may be used as a substitute to fulfill the functions of Vitamin E in this coating.

In accordance with one aspect of the present invention, the therapeutic agent can be mixed with a solvent prior to combining with the oil-based composition. The solvent can be a solvent compatible with the oil composition, therapeutic agent, and intended use.

In accordance with one aspect of the present invention, the therapeutic agent is dissolved in the oil-based composition, is a solid suspended in the oil-based composition, or a combination thereof.

In accordance with one aspect of the present invention, the viscosity measurement of the oil-based composition containing a therapeutic agent can be between about 5 cPs to about 150,000 cPs. In one embodiment, the viscosity measurement of the oil-based composition can be between about 30 cPs and about 30,000 cPs.

In accordance with one aspect of the present invention, the coating is non-polymeric. In accordance with one aspect of the present invention the coating can inhibit restenosis and neointimal growth. In accordance with one aspect of the present invention, the coating can promote endothelialization. In accordance with one aspect of the present invention, the coating is bio-absorbable.

In accordance with one aspect of the present invention, the release of the one or more therapeutic agents is extended by the increased viscosity of the oil-based composition. In accordance with another aspect of the present invention, the increased viscosity of the oil-based composition prevents the removal of the coating from a medical device in vivo. In accordance with one aspect of the present invention, the oil-based composition retains an anti-inflammatory or non-inflammatory characteristic.

In accordance with one aspect of the present invention, the medical device can be a stent, a mesh or a stand alone film. In various embodiments, the stent is formed of a substance selected from the group consisting of stainless steel, Nitinol alloy, nickel alloy, titanium alloy, cobalt-chromium alloy, tantalum, magnesium, ceramics, metals, plastics, and polymers.

### BRIEF DESCRIPTION OF THE DRAWINGS

The aforementioned features and advantages, and other features and aspects of the present invention, will become better understood with regard to the following description and accompanying drawings, wherein:

FIG. 1 is a flow chart illustrating a method of increasing the viscosity of an oil-based composition, in accordance with one embodiment of the present invention;

FIG. 2 is a flow chart illustrating a method of increasing the viscosity of an oil-based composition, in accordance with one embodiment of the present invention;

FIG. 3 is a flow chart illustrating a method of making a coating for a medical device, in accordance with one embodiment of the present invention;

FIG. 4 is a flow chart illustrating a method of making the coated medical device of the present invention, in accordance with one embodiment of the present invention;

FIG. 5 is a diagrammatic illustration of a medical device, according to one embodiment of the present invention;

FIG. 6 is a cross-sectional view of the medical device in accordance with one aspect of the present invention;

FIG. 7 is a cross-sectional view of the medical device in accordance with another aspect of the present invention;

FIG. 8 is a flow chart illustrating a variation of the method of FIG. 7, in accordance with one embodiment of the present invention;

FIG. 9 is a flow chart illustrating a variation of the method of FIG. 7, in accordance with one embodiment of the present invention;

FIG. 10 is a diagrammatic illustration of a coated medical device in accordance with one embodiment of the present invention;

FIG. 11 diagrammatic illustration of a barrier layer realized as a stand alone film, according to one embodiment of the present invention;

FIG. 12 is cross-sectional views of the barrier layer in accordance with one aspect of the present invention;

FIGS. 13A and 13B are perspective and cross-sectional views of the barrier layer in combination with a medical device, in accordance with one embodiment of the present invention; and

FIGS. 14A, 14B, and 14C are diagrammatic illustrations of the barrier coupled with various medical devices.

### DETAILED DESCRIPTION

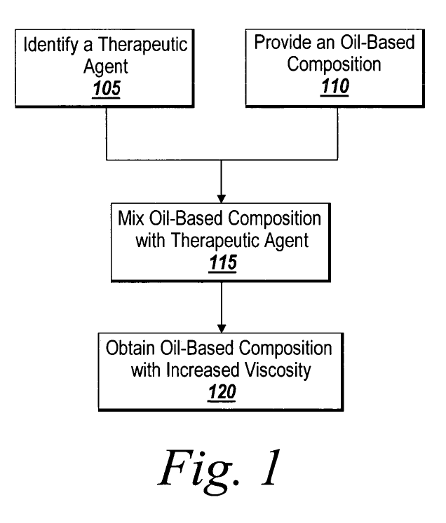

FIGS. 1 through 14C, wherein like parts are designated by like reference numerals throughout, illustrate examples of embodiments of increasing the viscosity of an oil-based composition and of embodiments of a coated medical device according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

FIG. 1 is a flow chart illustrating a method of the present invention, in the form of increasing the viscosity of an oil-based composition. In accordance with one aspect of the present invention, a therapeutic agent is identified (step 105). The therapeutic agents suitable for use in the invention are not particularly limited. The therapeutic agents can be hydrophilic, lipophilic, amphiphilic or hydrophobic. The therapeutic agent can be any agent having therapeutic value when administered to a subject, for example, a mammal. The therapeutic agent component can take a number of different forms including but not limited to anti-oxidants, anti-inflammatory agents, analgesics, anti-coagulant agents, drugs to alter lipid metabolism, anti-proliferatives, anti-neoplastics, tissue growth stimulants, functional protein/factor delivery agents, anti-infective agents, anti-imaging agents, anesthetic agents, therapeutic agents, tissue absorption enhancers, anti-adhesion agents, germicides, antiseptics, proteoglycans, GAG's, gene delivery (polynucleotides), polysaccharides (e.g. heparin), anti-migratory agents, pro-healing agents, and ECM/protein production inhibitors, rapamycin, melatonin, paclitaxel, a protein kinase C inhibitor, cerivastatin, cilostazol, fluvastatin, lovastatin, analgesics, pravastatin or derivatives, analogs, prodrugs and pharmaceutically acceptable salts thereof, and any additional desired therapeutic agents such as those listed in Table 1 below.

TABLE #1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Anti-inflammatory Agents | Glucocorticoids (e.g. dexamethazone, |
| | methylprednisolone), leflunomide, NSAIDS, ibuprofen, |
| | acetaminophen, hydrocortizone acetate, hydrocortizone |
| | sodium phosphate, macrophage-targeted bisphosphonates |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | micophenonolic acid, rapamycin, everolimus, tacrolimus, |
| | paclitaxel, QP-2, actinomycin, estradiols, dexamethasone, |
| | methatrexate, cilostazol, prednisone, cyclosporine, |
| | doxorubicin, ranpirnas, troglitzon, valsarten, pemirolast, C- |
| | MYC antisense, angiopeptin, vincristine, PCNA ribozyme, |
| | 2-chloro-deoxyadenosine |
| Tissue growth stimulants | Bone morphogeneic protein, fibroblast growth factor |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estradiols, nitric oxide, |
| delivery | endothelial progenitor cell antibodies |
| Second messenger | Protein kinase inhibitors |
| targeting | |
| Angiogenic | Angiopoetin, VEGF |
| Anti-Angiogenic | Endostatin |
| Inhibitation of Protein | Halofuginone, prolyl hydroxylase inhibitors, C-proteinase |
| Synthesis/ECM formation | inhibitors |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride, Selenium. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donor | NCX 4016 - nitric oxide donor derivative of aspirin, |
| Derivatives | SNAP |
| Gases | Nitric oxide, compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochlorite |
| Anti-Fibrotic Agents | Interferon gamma -1b, Interluekin -10 |
| Immunosuppressive/Immunomodulatory | Cyclosporine, rapamycin, mycophenolate motefil, |
| Agents | leflunomide, tacrolimus, tranilast, interferon gamma-1b, |
| | mizoribine |
| Chemotherapeutic Agents | Doxorubicin, paclitaxel, tacrolimus, sirolimus, fludarabine, |
| | ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyaluronic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |
| Antiseptics | Selenium |
| Analgesics | Bupivicaine, naproxen, ibuprofen, acetylsalicylic acid |

Some specific examples of therapeutic agents useful in the anti-restenosis realm include cerivastatin, cilostazol, fluvastatin, lovastatin, paclitaxel, pravastatin, rapamycin, a rapamycin carbohydrate derivative (for example as described in US Patent Application Publication 2004/0235762), a rapamycin derivative (for example as described in U.S. Pat. No. 6,200,985), everolimus, seco-rapamycin, seco-everolimus, and simvastatin.

In accordance with one embodiment of the present invention, the amount of the therapeutic agent to be added to the oil-based composition can be an amount up to the maximum amount that can be dissolved in the oil component. The maximum amount of the therapeutic agent that can be dissolved is readily determined by simple mixing, as the presence of any non-dissolved therapeutic agent is apparent after solvent removal on visual inspection. Other suitable techniques for inspection for the presence of crystal formation include, for example, visual inspection, microscopic inspections, as well as chemical analysis techniques such as scanning electron microscopy (SEM), environmental scanning electron microscopy (ESEM), differential scanning calorimetry (DSC) and atomic force microscopy (AFM). In various embodiments, the amount of the therapeutic agent will be less than the maximum that can be dissolved. In another embodiment, the amount of the therapeutic agent added to the oil-composition will be more than the maximum that can be dissolved.

The amount of the therapeutic agent in the present invention, in one embodiment, can be an effective amount. The term “effective amount” as used herein, refers to that amount of a compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, an effective amount refers to that ingredient alone. When applied to a combination, an effective amount can refer to combined amounts of the active ingredients that result in the therapeutic effect, whether administered in combination, serially or simultaneously. In various embodiments, where formulations comprise two or more therapeutic agents, such formulations can be described as an effective amount of compound A for indication A and an effective amount of compound B for indication B, such descriptions refer to amounts of A that have a therapeutic effect for indication A, but not necessarily indication B, and amounts of B that have a therapeutic effect for indication B, but not necessarily indication A. In a further embodiment, the one of therapeutic agents may have a synergistic effect on another therapeutic agent in a combination of therapeutic agents. Moreover, each therapeutic agent may have a synergistic effect on any other therapeutic agent provided in the invention. As used herein, “synergy” or “synergistic effect” refers to an enhancement of the therapeutic properties of one or more therapeutic agents of the invention. Furthermore two or more compounds may be administered for the same or different indication with or without a true synergism. In another embodiment, compound A can have an enhancement effect on compound B and compound B can have an enhancement effect on compound A. In another embodiment, A and B may have no effect upon each other.

It should be noted that using a therapeutic agent to increase the viscosity of an oil-based composition for use as a coating for a medical device has several benefits, for example, extending the release of a therapeutic agent, preventing the coating from being washed away in-vivo and providing coatings with samples with increased drug loading. The increased viscosity of the coating can allow an thicker layer of coating to be applied to the medical device. Furthermore, there can be therapeutic agent dissolved in the coating as well as suspended in the coating as a solid. In one embodiment, the oil-based composition can be mixed with one therapeutic agent to increase the viscosity of the composition, while a second therapeutic agent can be dissolved or suspended in the oil-based composition. Further uses of the oil-based composition can include more readily providing multi-layered coatings, as

Actual dosage levels of the active ingredients in a therapeutic formulation of the present invention may be varied so as to obtain an amount of the active ingredients which is effective to achieve the desired therapeutic response without being unacceptably toxic. The selected dosage level will depend upon a variety of pharmacokinetic factors including the activity of the particular therapeutic formulations of the present invention employed, or the ester, salt or amide thereof, the route of administration, the time of administration, the duration of administration, the rate of excretion of the particular compounds being employed, the duration of the treatment, other drugs, compounds and/or materials used in combination with the particular compounds employed, and like factors well known in the medical arts.

Some specific examples of therapeutic agents useful in the anti-restenosis realm include cerivastatin, cilostazol, fluvastatin, lovastatin, paclitaxel, pravastatin, rapamycin, and simvastatin.

Referring again to FIG. 1, an oil-based composition is provided (step 110). The terms “oil-based composition” and “oil composition” as used herein refer to a composition comprising a naturally occurring oil, fish oil fatty acids, fatty acid esters, free fatty acids, triglycerides, diglycerides, monoglycerides, partially hydrolyzed oil, oxidized oil or a combination thereof. In one embodiment, the naturally occurring oil is fish oil. Suitable fish oils can be obtained, for example from a variety of fish and can include cod liver oil, shark liver oil and fish body oils. In various embodiments, the components of fish oil include triacylglycerol, diacylglycerol, monoacylglycerol, phospholipids, sterylesters, sterols, fatty acid esters and free fatty acids. The quantities of total lipids may vary between different fish oils. In various embodiments, the fish oil is modified to a state of increased viscosity. The modification of the fish oil may be accomplished by techniques known to those skilled in the art. In addition, the oil-based composition has anti-inflammatory or non-inflammatory properties.

The term “fatty acid” as used herein refers to compounds comprising carbon, hydrogen and oxygen arranged as a carbon skeleton with a carboxyl group at one end. Saturated fatty acids have all hydrogens, thus have no double bonds. Monounsaturated fatty acids have one double bond and polyunsaturated fatty acids have more than one double bond. Examples of common fatty acids are seen in Table 2.

TABLE 2

| | # of Carbon | # of Double | | |
|---|---|---|---|---|
| Common Name | Atoms | Bonds | Scientific Name | Sources |
| Butyric acid | 4 | 0 | Butanoic acid | Butterfat |
| Caproic acid | 6 | 0 | Hexanoic acid | Butterfat |
| Caprylic acid | 8 | 0 | Octanoic acid | Coconut oil |
| Capric acid | 10 | 0 | Decanoic acid | Coconut oil |
| Lauric acid | 12 | 0 | Dodecanoic acid | Coconut oil |
| Myristic acid | 14 | 0 | Tetradecanoic acid | Palm kernel |
| | | | | oil |
| Palmitic acid | 16 | 0 | Hexadecanoic acid | Palm oil |
| Palmitoleic acid | 16 | 1 | 9-hexadecenoic acid | Animal fats |
| Stearic acid | 18 | 0 | Octadecanoic acid | Animal fats |
| Oleic acid | 18 | 1 | 9-octadecenoic acid | Olive oil |
| Vaccenic acid | 18 | 1 | 11-octadecenoic acid | Butterfat |
| Linoleic acid | 18 | 2 | 9,12-octadecadienoic | Safflower oil |
| | | | acid | |
| Alpha-linoleic acid | 18 | 3 | 9,12,15- | Flaxseed |
| | | | octadecatrienoic acid | |
| Gamma-linoleic | 18 | 3 | 6,9,12-octadecatrienoic | Borage oil |
| acid | | | acid | |
| Arachidic acid | 20 | 0 | Eicosanoic acid | Peanut oil, |
| | | | | fish oil |
| Gadoleic acid | 20 | 1 | 9-eicosenoic acid | Fish oil |
| Arachidonic acid | 20 | 4 | 5,8,11,14- | Liver fats |
| | | | eicosatetraenoic acid | |
| EPA | 20 | 5 | 5,8,11,14,17- | Fish oil |
| | | | eicosapentaenoic acid | |
| Behenic acid | 22 | 0 | Docasanoic acid | Rapeseed oil |
| Erucic acid | 22 | 1 | 13-doxosenoic acid | Rapeseed oil |
| DHA | 22 | 6 | 4,7,10,13,16,19- | Fish oil |
| | | | docosahexaenoic acid | |
| Lignoceric acid | 24 | 0 | Tetraxosanoic acid | Small |
| | | | | amounts in |
| | | | | most fats |

Polyunsaturated fats can be further broken down into omega-3 fatty acids and omega-6 fatty acids. Omega-3 and omega-6 fatty acids are also known as essential fatty acids because they are important for maintaining good health, despite the fact that the human body cannot make them on its own. As such, omega-3 and omega-6 fatty acids must be obtained from external sources, such as food. Omega-6 fatty acids can be characterized as linoleic acids, gamma-linoleic acids and arachidonic acid. Omega-3 fatty acids can be further characterized as eicosapentaenoic acid (EPA), docosahexanoic acid (DHA), and alpha-linolenic acid (ALA). Both EPA and DHA are known to have anti-inflammatory effects and wound healing effects within the human body.

As used herein, the term “fish oil fatty acids” refers to those fatty acids which can be obtained from fish oil. Fish oil fatty acids can include, but are not limited to, arachidic acid, gadoleic acid, arachidonic acid, eicosapentaenoic acid, docosahexaenoic acid, derivatives, analogs, pharmaceutically acceptable salts, and combinations thereof.

As used herein, the term “free fatty acids” refers to those fatty acids which are not bound to other molecules. Bound fatty acids can be bound to compounds including, but not limited to, glycerides, glycerophospatides, glycosyldiglycerides, sterol esters, waxes, acylglycerols, cholesterol esters and glycospingolipids. Free fatty acids can be derived from their bound form by techniques well known in the art, such as saponification. Suitable free fatty acids can include butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, and derivatives, analogs and pharmaceutically acceptable salts thereof. In various embodiments, free fatty acids can also comprise fish oil fatty acids.

In one aspect of the present invention, the oil-based composition is bio-absorbable. The term “bio-absorbable” as used herein generally refers to having the property or characteristic of being able to penetrate the tissue of a subject's body. In certain embodiments of the present invention, bio-absorption occurs through a lipophilic mechanism. The bio-absorbable substance is soluble in the phospholipid bi-layer of cells of body tissue, and therefore impacts how the bio-absorbable substance penetrates into the cells. In various embodiments, the bio-absorbable carrier can be bio-compatible. The term “bio-compatible” refers to materials that do not elicit a toxic or severe immunological response.

It should be noted that a bio-absorbable substance differs from a biodegradable substance. Biodegradable is generally defined as capable of being decomposed by biological agents, or capable of being broken down by microorganisms or biological processes. Biodegradation thus relates to the breaking down and distributing of a substance through the subject's body, verses the penetration of the cells of the subject's body tissue. Biodegradable substances can cause inflammatory response due to either the parent substance or those formed during breakdown, and they may or may not be absorbed by tissues.

In further detail, the term “bio-absorbable” generally refers to having the property or characteristic of being able to penetrate the tissues of a patient's body. In example embodiments of the present invention, the bio-absorbable coating contains lipids, many of which originate as triglycerides. It has previously been demonstrated that triglyceride products such as partially hydrolyzed triglycerides and fatty acid molecules can integrate into cellular membranes and enhance the solubility of drugs into the cell. Whole triglycerides are known not to enhance cellular uptake as well as partially hydrolyzed triglycerides, because it is difficult for whole triglycerides to cross cell membranes due to their relatively large molecule size. The vitamin E compound can also integrate into cellular membranes resulting in decreased membrane fluidity and cellular uptake.

It is also known that damaged vessels undergo oxidative stress. A composition containing an antioxidant such as alpha-tocopherol may aid in preventing further damage by this mechanism.

Referring again to FIG. 1, the oil-based composition and the identified therapeutic agent are mixed together (step 1115). Suitable mixing techniques include, for example, vortexing, sonicating, stirring, rolling, or shaking, or other methods of mixing well known in the art. Upon mixing, the therapeutic agent is substantially dissolved in the oil-based composition, is a solid suspended in the oil-based composition, or a combination thereof.

Referring again to FIG. 1, the oil-based composition in combination with the therapeutic agent results in an oil-based composition with an increased viscosity (step 120). As used herein, the term “viscosity” refers to the resistance of a fluid to shear or flow, and is a measure of the fluids adhesive/cohesive or frictional properties. This resistance is caused by intermolecular friction exerted when layers of fluids attempts to slide by an other. One of ordinary skill in the art would be readily able to measure the viscosity of the oil-based composition by using, for example, a viscometer. The term “increased viscosity” refers to an increase in the resistance of a fluid to shear or flow, as compared to a reference fluid. The units of viscosity can be centipoises (cP), centistokes (cSt), Saybolt Universal Seconds (SSU), Pascal seconds (Pa-s) and degrees Engler. In one embodiment, the oil-based composition of the oil-based composition has a viscosity measurement from about 50 cPs to about 30,000 cPs. Accordingly, oil-based composition can have a viscosity of about 90 cPs, of about 180 cPs, of about 700 cPs, of about 11,000 cPs, of about 20,000 cPs or about 28,000 cPs.

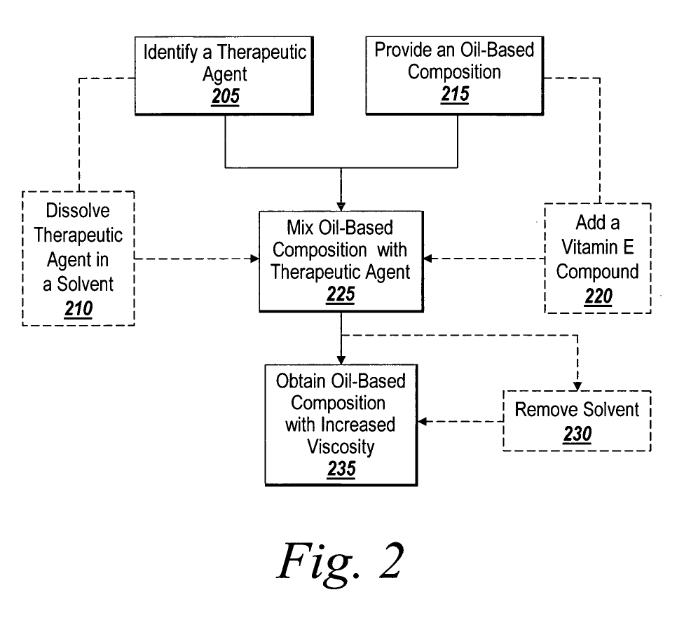

FIG. 2 is flow chart illustrating a method of the present invention, in the form of increasing the viscosity of an oil-based composition. In accordance with one aspect of the present invention, a therapeutic agent is identified (step 205). In one embodiment, the therapeutic agent can be dissolved in a solvent (step 210). The use of a solvent to dissolve the therapeutic agent is not always needed. In one embodiment, the therapeutic agent is dissolved in the oil-based composition without a solvent. In another embodiment, the therapeutic agent is suspended in the oil-based composition without the use of a solvent.

The solvent can be selected based on the identified therapeutic agent. One skilled in the art will be able to determine the appropriate solvent to use. The solvent can be a solvent or mixture of solvents and include solvents that are generally acceptable for pharmaceutical use. Suitable solvents include, for example: alcohols and polyols, such as C₂-C₆ alkanols, 2-ethoxyethanol, ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, and polypropylene glycol; amides, such as 2-pyrrolidone, 2-piperidone, 2-caprolactam, N-alkylpyrrolidone, N-methyl-2-pyrrolidone, N-hydroxyalkylpyrrolidone, N-alkylpiperidone, N-alkylcaprolactam, dimethylacetamide; esters, such as ethyl acetate, methyl acetate, butyl acetate, ethylene glycol diethyl ether, ethylene glycol dimethyl ether, propylene glycol dimethyl ether, ethyl proprionate, tributylcitrate, acetyl triethylcitrate, acetyl tributyl citrate, triethylcitrate, ethyl oleate, ethyl caprylate, ethyl cutyrate, tracetin, ε-caprolactone and isomers thereof, δ-valerolactorne and isomers thereof, β-butyrolactone and isomers thereof; and other solvents, such as water, dimethylsulfoxide, benzyl benzoate, ethyl lactate, acetone, methylethyl ketone, dimethylsolfone, tetrahydrofuran, decylmethylsufoxide, N,N-diethyl-m-toulamide or 1-dodecylazacycloheptan-2-one, hexane, chloroform, dichloromethane. Suitable solubility enhancers can include, for example, polyvinylalcohol, hydroxypropyl methylcellulose, and other celluloses, cyclodextrins and cyclodextrin derivatives.

The amount of solvent that can be included in compositions of the present invention is not particularly limited. Upon administration to a subject of the therapeutic agent dissolved in the bio-absorbable carrier and the solvent, the amount of the given solvent can be limited to a pharmaceutically acceptable amount, which can be readily determined by one of skill in the art. In various aspects, it can be appropriate to include amounts of solvents in excess of pharmaceutically acceptable amounts, with excess solvent removed prior to providing the administration of the composition using conventional techniques such as evaporation.

Referring again to FIG. 2, an oil-based composition is provided (step 215). In one embodiment, a vitamin E compound can be added to the oil-based composition (step 220). Vitamin E describes a family of eight fat-soluble antioxidants, the four tocopherols, alpha-, beta-, gamma- and delta- (Formula I), and the four tocotrienols also alpha-, beta-, gamma- and delta- (Formula II):

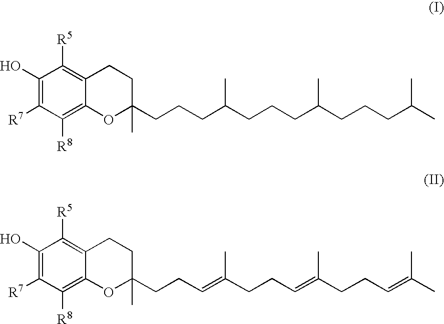

| Tocopherol Structure | Tocotrienol Structure | R⁵, R⁷, R⁸ |
|---|---|---|
| Alpha-tocopherol | Alpha-tocotrienol | R⁵, R⁷, R⁸ = CH₃ |
| Beta-tocopherol | Beta-tocotrienol | R⁵, R⁸ = CH₃; R⁷ = H |
| Gamma-tocopherol | Gamma-tocotrienol | R⁷, R⁸ = CH₃; R⁵ = H |
| Delta-tocopherol | Delta-tocotrienol | R⁵, R⁷ = H; R⁸ = CH₃ |

The term “vitamin E compound” as used herein generally refers to any compound of the vitamin E family, including derivatives, analogs, and pharmaceutically acceptable salts thereof. The vitamin E compound and include, for example, alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, Vitamin E TPGS, mixed tocoherols, derivatives, analogs, pharmaceutically acceptable salts and mixtures thereof. Suitable vitamin E compound analogs can be, for example, desmethyl-tocotrienol, didesmethyl-tocotrienol, P₁₈ tocotrienol™, P₂₅ tocotrienol, alpha-tocomonoenol. The vitamin E compounds can be conveniently isolated from biological materials or synthesized from commercially available starting materials by techniques known to those skilled in the art. In various embodiments, the vitamin E compounds can be in their isomerically pure form or be present as mixtures of isomers. For example, the vitamin E compounds can exist as the D-isomer, the L-isomer, or the D,L-racemic mixture.

In one embodiment, other fat soluble vitamins can be used in the invention. Suitable fat soluble vitamins include, for example, vitamin A, vitamin D, vitamin K, and derivatives, pharmaceutically acceptable salts, esters and amides thereof.

The ratio of the vitamin E compound to the oil composition can be determined by techniques known to those skilled in the art. Accordingly, the oil composition with the vitamin E compound can be about 70% of an oil composition and about 30% of a vitamin E compound; about 70% of a vitamin E compound and about 30% of an oil composition; or about 50% of a vitamin E compound and about 50% of an oil composition.

In accordance with one aspect of the present invention, the oil composition and the vitamin E compound can be mixed together, for example, by vortexing, sonicating, stirring, rolling, or shaking or other methods of mixing well known in the art.

Referring again to FIG. 2, the oil-based composition, with or without the vitamin E compound, and the identified therapeutic agent, with or without the solvent, are mixed together (step 225). Suitable mixing techniques include, for example, vortexing, sonicating, stirring, rolling, or shaking. Upon mixing, the therapeutic agent is substantially dissolved in the oil-based composition, is a solid suspended in the oil-based composition, or a combination thereof. If a solvent is used, the solvent can be removed by techniques well known in the art, for example, by vacuum, heat, washing, evaporation and the like (step 230). Upon removal of the solvent, the resulting solution can be inspected for presence of crystal formation by techniques well known in the art.

Suitable techniques for inspection for the presence of crystal formation include, for example, visual inspection, microscopic inspections, as well as chemical analysis techniques such as scanning electron microscopy (SEM), environmental scanning electron microscopy (ESEM), differential scanning calorimetry (DSC) and atomic force microscopy (AFM).

Referring again to FIG. 2, the oil-based composition in combination with the therapeutic agent results in an oil-based composition with an increased viscosity (step 235).

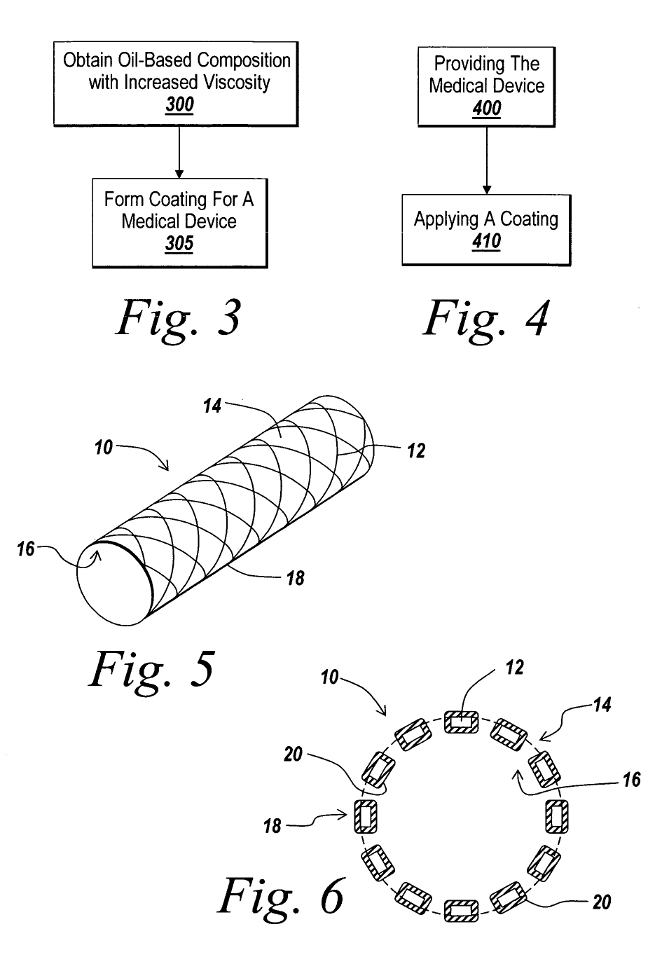

FIG. 3 is a flowchart illustrating a method of the present invention, in the form preparing a coating for medical devices, in accordance with one embodiment of the present invention. An oil-based composition with increased viscosity is first obtained as described above (step 300). The oil-based composition with increased viscosity forms the coating for a medical device (step 305).

In accordance with one aspect of the present invention, a coated medical device is provided. The medical devices of the invention can be, for example, a mesh or a stand alone film, a catheter, a guidewire, a cannula, a stent, a vascular or other graft, a cardiac pacemaker lead or lead tip, a cardiac defibrillator lead or lead tip, a heart valve, or an orthopedic device, appliance, implant, or replacement. In one aspect, the medical device is a stent. The term “stent” refers to what is known in the art as a metallic or polymeric cage-like device that is used to hold bodily vessels, such as blood vessels, open.

The device and methods of the present invention can be useful in a wide variety of locations within a human or veterinary patient, such as in the esophagus, trachea, colon, biliary tract, urinary tract and vascular systems, including coronary vessels, as well as for subdural and orthopedic devices, implants or replacements. They can be advantageous for reliably delivering suitable bioactive materials during or following an intravascular procedure or surgery, and find particular use in preventing abrupt closure and/or restenosis of a blood vessel. More particularly, they permit, for example, the delivery of an effective amount of one or more therapeutic agents to the region of a blood vessel which has been opened by PTA. The coated medical devices of the invention can be implantable in a subject. As used here, the term “subject” includes animals, (e.g., vertebrates, amphibians, fish) mammals (e.g., cats, dogs, horses, pigs, cows, sheep, rodents, rabbits, squirrels, bears) and primates (e.g., chimpanzees, gorillas, and humans).

The device of the present invention can be formed of a substance selected from the group consisting of stainless steel, nickel, silver, platinum, gold, titanium, tantalum, iridium, tungsten, Nitinol, inconel, Nitinol alloy, nickel alloy, titanium alloy, cobalt-chromium alloy, magnesium, tantalum, ceramics, metals, plastics, and polymers or the like.

FIG. 4 illustrates one method of making the present invention, in the form of the coated stent, in accordance with one embodiment of the present invention. The process involves providing a medical device, such as a stent (step 400). A coating, such as is then applied to the medical device (step 410). One of ordinary skill in the art will appreciate that this basic method of application of a coating to a medical device such as the stent can have a number of different variations falling within the process described. Depending on the particular application, the stent with the coating applied thereon can be implanted after the coating is applied, or additional steps such as curing and sterilization can be applied to further prepare the stent and coating. Furthermore, if the coating includes a therapeutic agent that requires some form of activation (such as UV light), such actions can be implemented accordingly.

FIG. 5 illustrates a stent 10 in accordance with one embodiment of the present invention. The stent 10 is representative of a medical device that is suitable for having a coating applied thereon to effect a therapeutic result. The stent 10 is formed of a series of interconnected struts 12 having gaps 14 formed therebetween. The stent 10 is generally cylindrically shaped. Accordingly, the stent 10 maintains an interior surface 16 and an exterior surface 18.

One of ordinary skill in the art will appreciate that the illustrative stent 10 is merely exemplary of a number of different types of stents available in the industry. For example, the strut 12 structure can vary substantially. The material of the stent can also vary from a metal, such as stainless steel, Nitinol, nickel, and titanium alloys, to cobalt chromium alloy, ceramic, plastic, and polymer type materials. One of ordinary skill in the art will further appreciate that the present invention is not limited to use on stents. Instead, the present invention has application on a wide variety of medical devices. For purposes of clarity, the following description will refer to a stent as the exemplary medical device. The terms medical device and stent are interchangeable with regard to the applicability of the present invention. Accordingly, reference to one or another of the stent, or the medical device, is not intended to unduly limit the invention to the specific embodiment described.

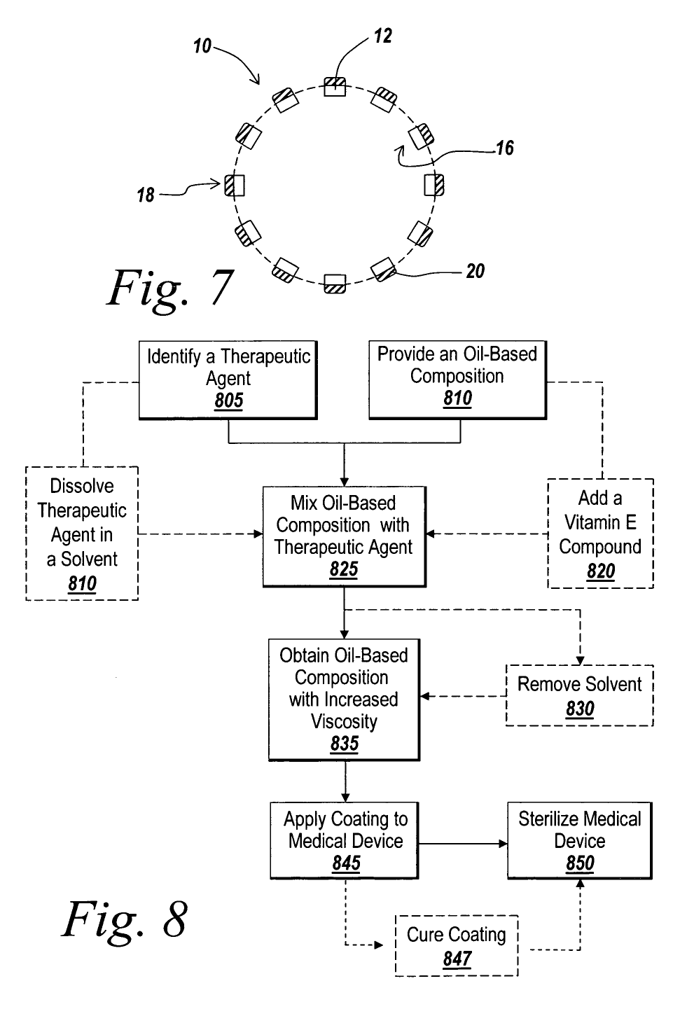

FIG. 6 illustrates one example embodiment of the stent 10 having a coating 20 applied thereon in accordance with the present invention. FIG. 7 is likewise an alternative embodiment of the stent 10 having the coating 20 also applied thereon. The coating 20 is applied to the medical device, such as the stent 10, to provide the stent 10 with different surface properties, and also to provide a vehicle for therapeutic applications.

In FIG. 6, the coating 20 is applied on both the interior surface 16 and the exterior surface 18 of the strut 12 forming the stent 10. In other words, the coating 20 in FIG. 6 substantially encapsulates the struts 12 of the stent 10. In FIG. 7, the coating 20 is applied only on the exterior surface 18 of the stent 10, and not on the interior surface 16 of the stent 10. The coating 20 in both configurations is the same coating; the difference is merely the portion of the stent 10 that is covered by the coating 20. One of ordinary skill in the art will appreciate that the coating 20 as described throughout the description can be applied in both manners shown in FIG. 6 and FIG. 7, in addition to other configurations partially covering select portions of the stent 10 structure. All such configurations are described by the coating 20 reference.

It should further be emphasized that the bio-absorbable nature of the coating results in the coating 20 being completely absorbed over time by the cells of the body tissue. The coating, or break down products of the coating, will not induce an inflammatory response. In short, the coating 20 is generally composed of fatty acids, including in some instances omega-3 fatty acids bound to trigycerides, and potentially also including a mixture of free fatty acids and vitamin E. The triglycerides are broken down by lipases (enzymes) which result in free fatty acids that can be transported across cell membranes. Subsequently, fatty acid metabolism by the cell occurs to metabolize any substances originating with the coating. The bio-absorbable nature of the coating of the present invention thus results in the coating being absorbed, leaving only an underlying delivery or other medical device structure. The oil-based composition does not induce a foreign body response, such as an inflammatory response The modification of the oils from a more liquid state to a more solid, but still flexible, physical state is implemented through a curing process. Curing with respect to the present invention generally refers to thickening, hardening, or drying of a material brought about by heat, UV, or chemical means. As the oils are cured, especially in the case of fatty acid-based oils such as fish oil, cross-links form creating a gel. As the curing process is performed over increasing time durations and/or increasing temperature conditions and/or increasing UV output, more cross-links form transitioning the gel from a relatively liquid gel to a relatively solid-like, but still flexible, gel structure.

The coatings for the medical device of the present invention can include an amount of one or more therapeutic agents dissolved and/or suspended in an oil-based composition with an increased viscosity. In one embodiment, the oil-based composition may contain a vitamin E compound, a solvent or both. The coatings of the invention can further contain a compatibilizer, a preservative or both. As used herein, the term “compatibilizer” refers to an added component of the coating that may prevent crystal formation after the removal of solvent. Suitable compatibilizers include, for example Vitamin E or its derivatives, free fatty acids, fatty acid esters, partially oxidized triglycerides, hydrolyzed triglycerides, therapeutic agents, antioxidants, surfactants and any amphiphilic materials. The term “preservative”, as used herein, refers to an added component of the coating that can prevent the deterioration of the therapeutic agent, the coating or both. Suitable preservatives include, for example, vitamin E or its derivatives, as well as antioxidant materials.

Accordingly, the coatings of the invention are non-polymeric As used herein, the term “polymer” is a generic term that is normally used by one of ordinary skill in the art to describe a substantially long molecule formed by the chemical union of five or more identical combining units called monomers. In most cases, the number of monomers is quite large (3500 for pure cellulose). See Hawley's Condensed Chemical Dictionary, page 900. Prior attempts to create drug delivery platforms such as coatings on stents primarily make use of polymer based coatings containing one or more therapeutic agents. Regardless of how much of the therapeutic agent would be most beneficial to the damaged tissue, the polymer releases the therapeutic agent based on the properties of the polymer coating. Accordingly, the effect of the coating is substantially local at the surface of the tissue making contact with the coating and the stent. In some instances, the effect of the coating is further localized to the specific locations of stent struts pressed against the tissue location being treated. These prior approaches can create the potential for a localized toxic effect. In addition, patients that received a polymer-based implant must also follow a course of long term systemic anti-platelet therapy, on a permanent basis, to offset the thrombogenic properties of the non-absorbable polymer. A significant percentage of patients that receive such implants are required to undergo additional medical procedures, such as surgeries (whether related follow-up surgery or non-related surgery) and are required to stop their anti-platelet therapy. This can lead to a thrombotic event, such as stroke, which can lead to death. Use of the inventive coating described herein can negate the necessity of anti-platelet therapy, and the corresponding related risks described, because there is no thrombogenic polymer reaction to the coating.

Due to the lipophilic mechanism enabled by the bio-absorbable coating 20 the uptake of the therapeutic agent is facilitated by the delivery of the therapeutic agent to the cell membrane by the oil-based composition. Further, the therapeutic agent is not freely released into the body fluids, but rather, is delivered directly to the cells and tissue. In prior configurations using polymer based coatings, the drugs were released at a rate regardless of the reaction or need for the drug on the part of the cells receiving the drug.

In addition, the bio-absorbable nature of the oil-based composition and the resulting coating results in the coating 20 being completely absorbed over time by the cells of the body tissue. The coating breaks down into sub-parts and substances which do not induce an inflammatory response and are eventually distributed through the body and, in some instances, disposed of by the body, as is the case with biodegradable coatings. The bio-absorbable nature of coating 20 of the present invention results in the coating being absorbed, leaving only the stent structure, or other medical device structure. There is no foreign body response to the bio-absorbable carrier component.

Despite the action by the cells, the coating 20 of the present invention can be further configured to release the therapeutic agent component at a rate no faster than a selected controlled release rate over a period of weeks to months. The controlled release rate action is achieved by providing an increased level of vitamin E in the mixture with the fish oil, to create a more viscous, sticky coating substance that better adheres and lasts for a longer duration on the implanted medical device. The controlled release rate can include an initial burst of release, followed by the sustained multi-week to multi-month period of release.

In addition, the oil provides a lubricious surface against the vessel walls. As the stent 10 having the coating 20 applied thereon is implanted within a blood vessel, for example, there can be some friction between the stent walls and the vessel walls. This can be injurious to the vessel walls, and increase injury at the diseased vessel location. The use of the naturally occurring oil, such as fish oil, to the surface of the stent 10, can reduce the initial injury. With less injury caused by the stent, there is less of an inflammatory response and less healing is required.

The coatings of the invention can inhibit restenosis, induced either biologically or mechanically. Biologically induced restenosis includes, but is not limited to injury attributed to infectious disorders including endotoxins and herpes viruses such as cytomegalovirus; metabolic disorders such as atherosclerosis; and vascular injury resulting from hypothermia, and irradiation. Mechanically induced restenosis includes, but is not limited to, vascular injury caused by catheterization procedures or vascular scraping procedures such as percutaneous transluminal coronary angioplasty; vascular surgery; transplantation surgery; laser treatment; and other invasive procedures which disrupt the integrity of the vessel.

The coatings of the invention can additionally inhibit neointimal growth. Neointimal growth refers to the migration and proliferation of vascular smooth muscle (VSM) cells with subsequent deposition of extracellular matrix components at the site of injury. Neointimal growth can occur as the result of arterial tissue injury caused by biological or mechanical origins. Injury can cause an exaggerated or excessive healing response characterized by excessive proliferation of the vascular smooth muscle cells in the neointima and subsequent secretion of extracellular matrix causing intimal hyperplasia that can often result in stenosis of the artery. While the mechanism is complex, the hyperplasia appears to result at least partly from transformation of the smooth muscle cells from a quiescent, contractile phenotype to a proliferative phenotype. If untreated the proliferation of cells and secretion of extracellular matrix can obstruct the vessel lumen.

The coatings of the invention can further promote endothelialization. Endothelialization refers to both any process of replacing the endothelium stripped by any biological or mechanical process and any process of growing new endothelial cells to cover an implanted medical device. The endothelialization can involve ingrowth of the proximal or distal endothelium longitudinally over the stent, from the lumen of the blood vessel into which the stent is inserted. Endothelialization via this method can result in endothelial cells lining the lumen of the stented vessel. Stents can be treated or coated with drugs or other substances which encourage endothelial growth and/or recruitment of endothelial progenitor cells for example from the blood circulation.

In the instance of an expanded PTFE vascular graft, covered stent or stent graft the endothelialization can involve promoting pannus ingrowth longitudinally into the device from the lumen of the blood vessel into which the stent is inserted. Endothelialization via this method can result in endothelial cells lining the lumen of the device with few if any endothelial cells in the porosity of the device. Endothelialization can also refer to “transmural” or “transinterstitial” endothelialization, which can involve promoting the ingrowth of capillaries and/or capillary endothelial cells through the device wall and into the porosity. Such endothelial cells originate in the microvasculature of adjacent tissue external to the device, and grow through the device wall, in part by virtue of its porosity. Under appropriate conditions, the endothelial cells are able to grow through the stent wall and colonize the stent lumen. Endothelialization can further refer to “capillary endothelialization”. The process of capillary endothelialization can be distinguished by its sequential cellular steps, including the initial attachment of endothelial cells to the stent material, followed by their spreading, inward migration, and optionally, proliferation. Accordingly, endothelialization can additionally refer to all of these processes. The term “endothelial cells” can refer to both mature endothelial cells and endothelial progenitor cells.

In accordance with one aspect of the present invention, the coatings can effect controlled delivery of the one or more therapeutic agents. The phrases “controlled release” and “delivery of the therapeutic agent is controlled” generally refers to the release of a biologically active agent in a predictable manner over the time period of, several days, several weeks or several months, as desired and predetermined upon formation of the biologically active agent on the medical device from which it is being released. Controlled release includes the provision of an initial burst of release upon implantation, followed by the predictable release over the aforementioned time period.

Furthermore, the step of applying a coating substance to form a coating on the medical device such as the stent 10 can include a number of different application methods. For example, the stent 10 can be dipped into a liquid solution of the coating substance. The coating substance can be sprayed onto the stent 10, which results in application of the coating substance on the exterior surface 18 of the stent 10 as shown in FIG. 7. Another alternative application method is painting, using an applicator or wiping the coating substance on to the stent 10, which also results in the coating substance forming the coating 20 on the exterior surface 18 as shown in FIG. 7. One of ordinary skill in the art will appreciate that other methods, such as electrostatic adhesion and inkjet application, and other application methods, can be utilized to apply the coating substance to the medical device such as the stent 10. Some application methods may be particular to the coating substance and/or to the structure of the medical device receiving the coating. Accordingly, the present invention is not limited to the specific embodiment described herein, but is intended to apply generally to the application of the coating substance to the medical device, taking whatever precautions are necessary to make the resulting coating maintain desired characteristics.

FIG. 8 is a flowchart illustrating one example implementation of the method of FIG. 7. In accordance with the steps illustrated in FIG. 8, the therapeutic agent desired for delivery is identified (step 805). A solvent based on the properties of the therapeutic agent can be selected to dissolve the therapeutic agent, if desired (step 810). An oil-based composition is also provided (step 815) and, in one embodiment, a vitamin E compound can be added to the oil-based composition (step 820). Mixing of the oil-based composition, with or without the vitamin E compound, and the therapeutic agent, with or without the solvent, can then occur (step 825). If a solvent has been utilized, the solvent can then be removed (step 830). Upon mixing, the oil-based composition is obtained with an increased viscosity (step 835) and the coating is applied to the medical device (step 845). In one embodiment, the coating can be cured (step 847). The coating for a medical device can take place in a manufacturing-type facility and subsequently shipped and/or stored for later use. Alternatively, the coating 20 can be applied to the stent 10 just prior to implantation in the patient. The process utilized to prepare the stent 10 will vary according to the particular embodiment desired. In the case of the coating 20 being applied in a manufacturing-type facility, the stent 10 is provided with the coating 20 and subsequently sterilized in accordance with any of the methods provided herein, and/or any equivalents. The stent 10 is then packaged in a sterile environment and shipped or stored for later use. When use of the stent 10 is desired, the stent is removed from the packaging and implanted in accordance with its specific design.

In the instance of the coating being applied just prior to implantation, the stent can be prepared in advance. The stent 10, for example, can be sterilized and packaged in a sterile environment for later use. When use of the stent 10 is desired, the stent 10 is removed from the packaging, and the coating substance is applied to result in the coating 20 resident on the stent 10. The coating 20 can result from application of the coating substance by, for example, the dipping, spraying, brushing, swabbing, wiping, printing, using an applicator or painting methods.

The coated medical device is then sterilized using any number of different sterilization processes (step 850). Sterilization can involve use of at least one of ethylene oxide, gamma radiation, e-beam, steam, gas plasma, and vaporized hydrogen peroxide (VHP).

One of ordinary skill in the art will appreciate that other sterilization processes can also be applied, and that those listed herein are merely examples of sterilization processes that result in a sterilization of the coated stent, preferably without having a detrimental effect on the coating 20.

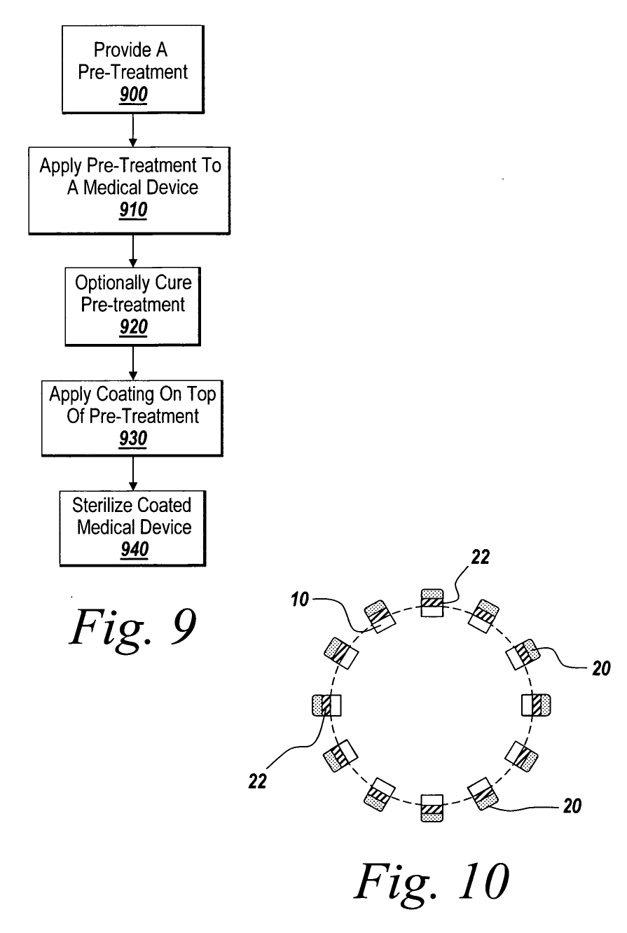

In accordance with another embodiment of the present invention a surface preparation or pre-treatment 22, as shown in FIG. 10, is provided on a stent 10. More specifically and in reference to the flowchart of FIG. 9, a pre-treatment substance is first provided (step 900). The pre-treatment substance is applied to a medical device, such as the stent 10, to prepare the medical device surface for application of the coating (step 910). Suitable pre-treatments include partially cured fish oil, reactive oils, plasma, parylene, and hydrophobic or hydrophilic polymers. If desired, the pre-treatment 22 is cured (step 920). Curing methods can include processes such as application of UV light or application of heat to cure the pre-treatment 22. A coating substance is then applied on top of the pre-treatment 22 (step 930). The coated medical device is then sterilized using any number of sterilization processes as previously mentioned (step 940).

FIG. 10 illustrates the stent 10 having two coatings, specifically, the pre-treatment 22 and the coating 20. The pre-treatment 22 serves as a base or primer for the coating 20. The coating 20 conforms and adheres better to the pre-treatment 22 verses directly to the stent 10, especially if the coating 20 is not heat or UV cured. The pre-treatment can be formed of a number of different materials or substances. In accordance with one example embodiment of the present invention, the pre-treatment is formed of a bio-absorbable substance, such as a naturally occurring oil (e.g., fish oil). The bio-absorbable nature of the pre-treatment 22 results in the pre-treatment 22 ultimately being absorbed by the cells of the body tissue after the coating 20 has been absorbed.

It has been previously mentioned that curing of substances such as fish oil can reduce or eliminate some of the therapeutic benefits of the omega-3 fatty acids, including anti-inflammatory properties and healing properties. However, if the coating 20 contains the oil-based composition having the therapeutic benefits, the pre-treatment 22 can be cured to better adhere the pre-treatment 22 to the stent 10, without losing the therapeutic benefits resident in the subsequently applied coating 20. Furthermore, the cured pre-treatment 22 provides better adhesion for the coating 20 relative to when the coating 20 is applied directly to the stent 10 surface. In addition, the pre-treatment 22, despite being cured, remains bio-absorbable, like the coating 20. In addition, methods can be used to enhance the curing process. These methods include, for example, the addition of other reactive oils, such as linseed oil, and the application of reactive gasses, such as oxygen, fluorine, methane or propylene, plasma treatment, and pressure in the presence of reactive gasses and the like.

The pre-treatment 22 can be applied to both the interior surface 16 and the exterior surface 18 of the stent 10, if desired, or to one or the other of the interior surface 16 and the exterior surface 18. Furthermore, the pre-treatment 22 can be applied to only portions of the surfaces 16 and 18, or to the entire surface, if desired. In one embodiment, the pre-treatment can include a therapeutic agent.

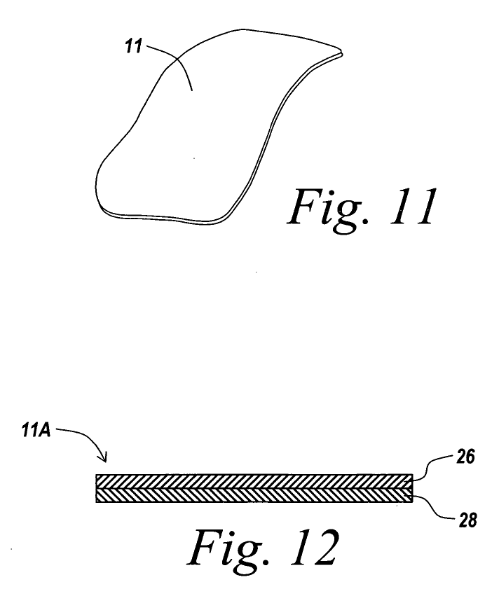

FIG. 11 illustrates a non-polymeric biological oil barrier layer 11 in accordance with one embodiment of the present invention. The barrier layer can be its own medical device (i.e., a stand alone film), or the barrier layer can be combined with another medical device to provide anti-adhesion characteristics, in addition to improved healing and delivery of therapeutic agents. The barrier layer is generally formed of a naturally occurring oil, or an oil composition formed in part of a naturally occurring oil. In addition, the oil composition can include a therapeutic agent component, such as a drug or other bioactive agent. The barrier layer is implantable in a patient for short term or long term applications, and can include controlled release of the therapeutic agent. As implemented herein, the barrier layer is a non-polymeric cross-linked gel derived at least in part from a fatty acid compound.

The barrier layer 11 is flexible, to the extent that it can be placed in a flat, curved, or rolled, configuration within a patient. The barrier layer 11 is implantable, for both short term and long term applications. Depending on the particular formulation of the barrier layer 11, the barrier layer 11 will be present after implantation for a period of hours to days, or possibly months.

FIG. 12 illustrates a side views of one embodiment of the barrier layer 11. In FIG. 12, a barrier layer 11A is shown having two tiers, a first tier 26 and a second tier 28. The first tier 26 and the second tier 28 as shown are formed of different materials. The different materials can be different forms of oil-based compounds. In one embodiment, the second tier can be a coating comprising an oil-based composition with increased viscosity. The different materials bind together to form the barrier layer 11A.

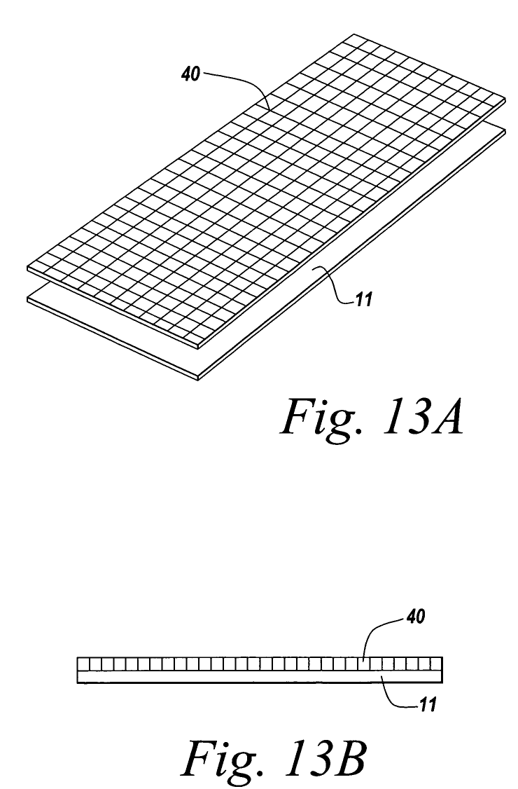

FIGS. 13A and 13B illustrate the barrier layer 11 and a medical device in the form of a mesh 40. In FIG. 13A, the barrier layer 11 and mesh 40 are shown in exploded view, while FIG. 13B shows the barrier layer 11 coupled with the mesh 40. The mesh 40 is merely one example medical device that can be coupled with the barrier layer 11. In the instance of the mesh 40, it can be useful to have one side of the mesh support a rougher surface to encourage tissue in-growth, and the other side of the mesh with an anti-adhesion, anti-inflammatory, and/or non-inflammatory surface to prevent the mesh from injuring surrounding tissue or causing inflammation. The coupling of the barrier layer 11 with the mesh 40 achieves such a device.

As understood by one of ordinary skill in the art, the properties of the mesh 40 and the barrier layer 11 can vary. There may be a requirement for the mesh 40 to have one side, or a portion of a side, that has anti-adhesion properties for a period of several days. Alternatively, multiple sides of the mesh 40 may be required to have anti-adhesion properties. As such, the barrier layer 11 can be applied to all sides, or portions of sides, or portions of one side of the mesh 40. In one embodiment, the mesh, the barrier layer or both can have a coating comprising an oil composition with increased viscosity.

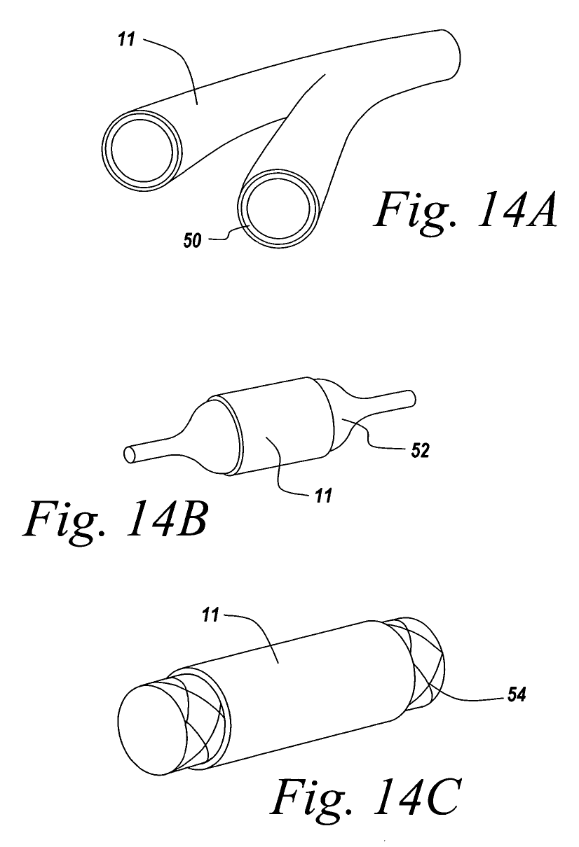

FIGS. 14A, 14B, and 14C illustrate some of the other forms of medical devices mentioned above in combination with the barrier layer 11 of the present invention. FIG. 14A shows a graft 50 with the barrier layer 11 coupled or adhered thereto. FIG. 14B shows a catheter balloon 52 with the barrier layer 11 coupled or adhered thereto. FIG. 14C shows a stent 54 with the barrier layer 11 coupled or adhered thereto. Each of the medical devices illustrated, in addition to others not specifically illustrated or discussed, can be combined with the barrier layer 11 using the methods described herein, or variations thereof. Accordingly, the present invention is not limited to the example embodiments illustrated. Rather the embodiments illustrated are merely example implementations of the present invention.

Various aspects and embodiments of the present invention are further described by way of the following Examples. The Examples are offered by way of illustration and not by way of limitation.

### EXAMPLE #1

An oil composition (Mixture A) was prepared by mixing 5 grams of fish oil with 5 grams of vitamin E. A therapeutic component was prepared by mixing 520 mg of rapamycin in 1690 mg of NMP and dissolving with a combination of vortexing and sonication to form mixture B. An amount of 1018 mg of mixture A was then added to mixture B and the two mixtures were combined by vortexing to form Mixture C. Mixture C was then placed in a 10 CC syringe and put onto a rotating fixture in a vacuum bell jar at a pressure of 50 mtorr for 50 hours. The resulting mixture D, which is a drug thickened version of Mixture A, had a final drug content of 33.8%. Mixture A and mixture D were then tested on a Physica MCR Rheometer and the viscosity was recorded at a sheer rate of 11/s. Mixture A was found to have a viscosity of 180 Cps and the drug thickened sample D was found to have a viscosity of 20,000 Cps.

### EXAMPLE #2

An oil composition (Mixture A) was prepared by mixing 1.5 grams of fish oil with 3.5 grams of vitamin E. A therapeutic component was prepared by mixing 759 mg of Cyclosporine in 777 mg of Ethanol and dissolving with a combination of vortexing and sonication to form mixture B. An amount of 1487 mg of mixture A was then added to mixture B and the two mixtures were combined by vortexing to form Mixture C. Mixture C was then placed in a 10 CC syringe and put onto a rotating fixture in a vacuum bell jar at a pressure of 50 mtorr for 50 hours. The resulting mixture D which is a drug thickened version of Mixture A had a final drug content of 33.8%. Mixture A and mixture D were then tested on a Physica MCR Rheometer and the viscosity was recorded at a sheer rate of 11/s. Mixture A was found to have a viscosity of 688 Cps and the drug thickened sample D was found to have a viscosity of 27,350 Cps.

### EXAMPLE #3

An oil composition (Mixture A) was prepared by mixing 1.5 grams of fish oil with 3.5 grams of vitamin E. A therapeutic component was prepared by mixing 77 mg of Cyclosporine in 1424 mg of Ethanol and dissolving with a combination of vortexing and sonication to form mixture B. An amount of 1433 mg of mixture A was then added to mixture B and the two mixtures were combined by vortexing to form Mixture C. Mixture C was then placed in a 10 CC syringe and put onto a rotating fixture in a vacuum bell jar at a pressure of 50 mtorr for 50 hours. The resulting mixture D, which is a drug thickened version of Mixture A, had a final drug content of 5.1%. Mixture A and mixture D were then tested on a Physica MCR Rheometer and the viscosity was recorded at a sheer rate of 11/s. Mixture A was found to have a viscosity of 688 Cps and the drug thickened sample D was found to have a viscosity of 11,080 Cps.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the appended claims is reserved. It is intended that the present invention be limited only to the extent required by the appended claims and the applicable rules of law.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

While the present inventions have been described in conjunction with various embodiments and examples, it is not intended that the present teachings be limited to such embodiments or examples. On the contrary, the present inventions encompass various alternatives, modifications, and equivalents, as will be appreciated by those of skill in the art.

The claims should not be read as limited to the described order or elements unless stated to that effect. It should be understood that various changes in form and detail may be made without departing from the scope of the appended claims. Therefore, all embodiments that come within the scope and spirit of the following claims and equivalents thereto are claimed.

Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

What is claimed is:

1. A method of increasing the viscosity of an oil-based composition, comprising:
providing the oil-based composition comprising at least one fatty acid; and
combining the oil-based composition with one or more therapeutic agents in an amount sufficient to increase viscosity of the oil based composition.

2. The method of claim 1, wherein the fatty acid comprises one or more of arachidic acid, gadoleic acid, arachidonic acid, eicosapentaenoic acid (EPA), docosahexaenoic acid (DHA), butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, analogs and pharmaceutically acceptable salts thereof.

3. The method of claim 1, wherein the therapeutic agent comprises an antioxidant, an anti-inflammatory, and anti-coagulant, a drug to alter lipid metabolism, an anti-proliferative, an anti-neoplastic, an anti-fibrotic, an immunosuppressive, a tissue growth stimulant, a functional protein/factor delivery agent, an anti-infective agent, an imaging agent, an anesthetic, a chemotherapeutic agent, a tissue absorption enhancer, an anti-adhesion agent, a germicide, an antiseptic, a proteoglycan, a GAG, a gene delivery agent (polynucleotide), an analgesic, a polysaccharide (heparin), or a derivative, an analog or a pharmaceutically acceptable salt thereof

4. The method of claim 1, wherein the therapeutic agent comprises one or more of rapamycin, melatonin, paclitaxel, cerivastatin, cilostazol, fluvastatin, lovastatin, pravastatin or derivatives, prodrugs, analogs and pharmaceutically acceptable salts thereof.

5. The method of claim 1, wherein the oil-based composition further comprises a vitamin E compound selected from the group consisting of alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, vitamin E TPGS, mixed tocopherols, derivatives, analogs and pharmaceutically acceptable salts thereof.

6. The method of claim 1, further comprising the step of mixing the one or more therapeutic agents with a solvent prior to combining the therapeutic agent with the oil-based composition.

7. The method of claim 6, wherein the solvent is selected from the group consisting of C₂-C₆ alkanols, 2-ethoxyethanol, ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, 2-pyrrolidone, 2-piperidone, 2-caprolactam, N-alkylpyrrolidone, N-methyl-2-pyrrolidone, N-hydroxyalkylpyrrolidone, N-alkylpiperidone, N-alkylcaprolactam, dimethylacetamide; ethyl acetate, methyl acetate, butyl acetate, ethylene glycol diethyl ether, ethylene glycol dimethyl ether, propylene glycol dimethyl ether, ethyl proprionate, tributylcitrate, acetyl triethylcitrate, acetyl tributyl citrate, triethylcitrate, ethyl oleate, ethyl caprylate, ethyl cutyrate, tracetin, ε-caprolactone and isomers thereof, δ-valerolactorne and isomers thereof, β-butyrolactone and isomers thereof; water, dimethylsulfoxide, benzyl benzoate, ethyl lactate, acetone, methylethyl ketone, dimethylsolfone, tetrahydrofuran, decylmethylsufoxide, N,N-diethyl-m-toulamide or 1-dodecylazacycloheptan-2-one, hexane, chloroform, dichloromethane, or a combination thereof.

8. The method of claim 1, wherein the therapeutic agent is dissolved in the oil-based composition without a solvent.

9. The method of claim 1, wherein the therapeutic agent is dissolved in the oil-based composition, is a solid suspended in the oil-based composition, or a combination thereof.

10. The method of claim 1, wherein the viscosity increases from about 5 cPs to about 150,000 cPs.

11. A coating for a medical device, comprising:
a composition formed at least in part of an oil comprising at least one fatty acid component and at least one therapeutic agent component;
wherein at least one therapeutic agent component is combined with the composition in an amount sufficient to increase the viscosity of the composition to a viscosity measurement greater than the viscosity measurement of the oil prior to combination with at least one therapeutic agent;
wherein the composition is configured for coating a medical device.

12. The coating of claim 11, wherein the at least one fatty acid comprises one or more of arachidic acid, gadoleic acid, arachidonic acid, eicosapentaenoic acid (EPA), docosahexaenoic acid (DHA), butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, analogs and pharmaceutically acceptable salts thereof.

13. The coating of claim 11, wherein at least one therapeutic agent comprises an antioxidant, an anti-inflammatory, an anti-coagulant, a drug to alter lipid metabolism, an anti-proliferative, an anti-neoplastic, an anti-fibrotic, an immunosuppressive, a tissue growth stimulant, a functional protein/factor delivery agent, an anti-infective agent, an imaging agent, an anesthetic, a chemotherapeutic agent, a tissue absorption enhancer, an anti-adhesion agent, a germicide, an antiseptic, a proteoglycan, a GAG, a gene delivery agent (polynucleotide), an analgesic, a polysaccharide (heparin), or a combination thereof.

14. The coating of claim 11, wherein the at least one therapeutic agent comprises one or more of rapamycin, melatonin, paclitaxel, cerivastatin, cilostazol, fluvastatin, lovastatin, pravastatin or derivatives, prodrugs, analogs and pharmaceutically acceptable salts thereof.

15. The coating of claim 11, wherein the oil-based composition further comprises a vitamin E compound selected from the group consisting of alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, vitamin E TPGS, mixed tocopherols, derivatives, analogs and pharmaceutically acceptable salts thereof.

16. The coating of claim 11, further comprising the step of mixing the one or more therapeutic agents with a solvent prior to combining the therapeutic agent with the oil-based composition.

17. The coating of claim 16, wherein the solvent is selected from the group consisting of C₂-C₆ alkanols, 2-ethoxyethanol, ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, 2-pyrrolidone, 2-piperidone, 2-caprolactam, N-alkylpyrrolidone, N-methyl-2-pyrrolidone, N-hydroxyalkylpyrrolidone, N-alkylpiperidone, N-alkylcaprolactam, dimethylacetamide; ethyl acetate, methyl acetate, butyl acetate, ethylene glycol diethyl ether, ethylene glycol dimethyl ether, propylene glycol dimethyl ether, ethyl proprionate, tributylcitrate, acetyl triethylcitrate, acetyl tributyl citrate, triethylcitrate, ethyl oleate, ethyl caprylate, ethyl cutyrate, tracetin, ε-caprolactone and isomers thereof, δ-valerolactorne and isomers thereof, β-butyrolactone and isomers thereof; water, dimethylsulfoxide, benzyl benzoate, ethyl lactate, acetone, methylethyl ketone, dimethylsolfone, tetrahydrofuran, decylmethylsufoxide, N,N-diethyl-m-toulamide or 1-dodecylazacycloheptan-2-one, hexane, chloroform, dichloromethane, or a combination thereof.

18. The coating of claim 11, wherein the at least one therapeutic agent is substantially dissolved in the oil-based composition, is a solid suspended in the oil-based composition or a combination thereof.

19. The coating of claim 11, wherein the oil-based composition has a viscosity measurement from about 50 cPs to about 150,000 cPs.

20. The coating of claim 11, wherein the medical device comprises a stent, a mesh, a graft, a balloon, a catheter or a stand alone film.

21. The coating of claim 11, wherein the coating inhibits restenosis.

22. The coating of claim 11, wherein the coating is non-polymeric.

23. The coating of claim 11, wherein the coating inhibits neo-intimal growth.

24. The coating of claim 11, wherein the coating promotes endothelialization.

25. The coating of claim 11, wherein release of the one or more therapeutic agents is extended by the increased viscosity of the oil-based composition.

26. The coating of claim 11, wherein the increased viscosity of the oil-based composition prevents the removal or reduces the amount of removal of the coating from a medical device in vivo.

27. The coating of claim 11, wherein the oil-based composition with increased viscosity retains an anti-inflammatory or non-inflammatory characteristic.
